(19) United States
(12) Patent Application Publication
(10) Pub. No.: US 2015/0082479 A1
(43) Pub. Date: Mar. 19, 2015

(54) CHEMICAL SELECTION OF RESISTANT GAMETES OF PLANTS IN THE FIELD

(71) Applicant: PIONEER HI BRED INTERNATIONAL INC, Johnston, IA (US)

(72) Inventors: Michael J. Lauer, Des Moines, IA (US); ROGER LEAFGREN, Woodland, CA (US); SCOTT NELSON, Urbandale, IA (US)

(21) Appl. No.: 14/552,529

(22) Filed: Nov. 25, 2014

Related U.S. Application Data
(63) Continuation of application No. 12/434,175, filed on May 1, 2009, now abandoned.
(60) Provisional application No. 61/049,816, filed on May 2, 2008.

(51) Int. Cl. C12N 15/82; A01H 1/04

(52) U.S. Cl. CPC C12N 15/8209; A01H 1/04; C12N 15/8275
USPC 800/272

(57) ABSTRACT

The present invention provides methods for chemical selection of female gametes that do not contain a transgene of interest in plants. The methods result is increased efficiency of a breeding program to provide increased trait purity by ensuring that inbreds and plants are homozygous for a transgene of interest. Herbicides such as glyphosate can be used as the chemical gametocide to selectively eliminate female gametes that do not contain the gene conferring glyphosate resistance, resulting in seed or progeny substantially homozygous for such herbicide resistance on a large scale in a single generation.

### CROSS-REFERENCE TO RELATED APPLICATIONS

This application claims priority to U.S. patent application Ser. No. 12/434,175 filed May 1, 2009, and to provisional application Ser. No. 61/049,816 filed May 2, 2008, herein incorporated by reference in their entirety.

### FIELD OF THE INVENTION

Embodiments of the present invention relate to the chemical selection of resistant gametes of plants in the field. These methods are particularly useful for increasing the efficiency of a breeding program through applying herbicides, such as glyphosate, to selectively eliminate female gametes that do not contain the gene conferring herbicide resistance. The methods result in a seed or progeny that is substantially homozygous for a transgene of interest, such as herbicide resistance.

### BACKGROUND OF THE INVENTION

The alteration of crop genetics to optimize agronomic traits has resulted in significant changes within the seed industry. Most agronomic traits are quantitative traits controlled by multiple genes, resulting in phenotype variability among the progeny. Heterozygous plants are not desirable for use as gene donors for producing commercial seed, as much of the seed lacks the desired trait, resulting in low levels of trait purity. Therefore, it is desirable to control the inheritance of such variable genes and resultant phenotypes for improved breeding efficiency and consistency.

A major limitation to the optimization of agronomic traits is that there have been no methods capable of ensuring large scale conversion of hemizygous transgenic plants to homozygous transgenic plants in a single generation. Prior methods have required breeders to self-pollinate and increase seed for several generations, selecting against susceptible plants at each stage by eliminating rows showing segregation for a transgenic trait. As a result, extensive labor and significant numbers of rows and generations were required to achieve homozygosity of transgenes. Consequently, under these methods, seed volumes become exceedingly high very quickly at the parent seed level (i.e., within one or two generations) and require breeders to discard the volumes of seed with insufficient transgene homozygosity. As a result of such practices, there is a need for methods and a process whereby seeds may increase from the hemizygous state to the homozygous state on a large scale and within a single generation.

Additionally, earlier methods of agronomy optimization required the selection of male gametes, as disclosed in U.S. Pat. No. 6,750,377 for methods of breeding glyphosate resistant plants, and in Touraev et al., Pollen Selection: A Transgenic Reconstruction Approach, 1995, Proc. Natl. Acad. Sci. USA, 92:12165-12169 for methods of male gametophytic selection. These methods failed to successfully provide plants with sufficient transmission of genes to a subsequent generation, within only one generation. As a result of such practices, there is a need for methods of selecting against females gametes not carrying the transgene of interest in order to form homozygous seeds within one generation.

Accordingly, an embodiment of the invention provides a method for reducing the number of rows and generations required to achieve homozygosity of transgenes.

A further embodiment of the invention provides a method of enabling the restoration of homozygosity in later generation seed increases which otherwise would have been of low or unacceptable transgene purity.

Yet another embodiment of the invention includes methods for improving inbred and hybrid seed production.

A still further embodiment of the invention provides a method of selecting against females gametes to ensure transgenetic purity in inbred line development.

The foregoing and other aspects of the present invention will become more apparent from the following description of the invention.

### SUMMARY OF THE INVENTION

This invention provides a novel means for the chemical selection of female gametes not containing a transgene of interest. Chemical selection of female gametes that do not contain a particular transgene in plants in a field results in increased efficiency of a breeding program. In one embodiment of the invention, a herbicide such as glyphosate is applied to selectively eliminate female gametes that do not contain the gene conferring herbicide resistance. This results in a seed or progeny substantially homozygous for such herbicide resistance. As a result, breeding by these methods allows the increasing of seeds from a hemizygous state to a homozygous state on a large scale and within a single generation, which was previously unachievable.

### BRIEF DESCRIPTION OF THE DRAWINGS

FIG. 1 shows application of herbicide at growth phase V12.

FIG. 2 shows application of herbicide at growth phase V17 and V19.

FIG. 3 shows application of herbicide at growth phase V17.

FIG. 4 shows application of herbicide at growth phase V15 and V17.

FIG. 5 shows application of herbicide at growth phase V19.

FIG. 6 shows glyphosate resistant plants treated with glyphosate at various rates and growth stages and crossed with wild type plants.

FIG. 7 shows the effect of glyphosate on pollen.

FIG. 8 shows the effect of glyphosate on ovules.

FIG. 9 shows the glyphosate resistance of seed and plant progeny.

FIG. 10 shows the testing of seed and plant progeny for scored levels of zygosity via QtPCR.

### DETAILED DESCRIPTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, processes and examples described in the description of the invention are illustrative only and not intended to be limiting to the scope of the invention in any manner. Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims.

The following definitions used in the specification and examples herein are provided below. In order to provide a clear and consistent understanding of the specification and claims, including the scope to be given such terms, the following definitions are provided:

As used herein, “agronomics,” “agronomic traits,” and “agronomic performance” refer to the traits (and underlying genetic elements) of a given plant variety that contribute to yield over the course of growing season. Individual agronomic traits include, but are not limited to, emergence vigor, vegetative vigor, stress tolerance, pest and disease resistance or tolerance, herbicide resistance, branching, flowering, seed set, seed size, seed density, standability, threshability and the like.

As used herein, “allele” refers to any of one or more alternative forms of a genetic sequence. Typically, in a diploid cell or organism, the two alleles of a given sequence typically occupy corresponding loci on a pair of homologous chromosomes.

As used herein, “alter” refers to the utilization of up-regulation, down-regulation, or gene silencing to change the expression level of a given gene.

As used herein, “breeding” refers to the genetic manipulation of living organisms.

As used herein, “breeding cross” refers to a cross to introduce new genetic material into a plant for the development of a new variety. For example, one could cross plant A with plant B, wherein plant B would be genetically different from plant A. After the breeding cross, the resulting F1 plants could then be selfed or sibbed for one, two, three or more times (F1, F2, F3, etc.) until a new inbred variety is developed. For clarification, such new inbred varieties would be within a pedigree distance of one breeding cross of plants A and B. The process described above would be referred to as one breeding cycle.

As used herein, the term “cross” or “crossing” can refer to a simple X by Y cross, or the process of backcrossing, depending on the context.

As used herein, “gamete” refers to a cell that fuses with another gamete during fertilization in organisms undergoing sexual reproduction. Many species, including plants, produce distinct types of gametes, wherein any individual plant produces only one type of gamete. In plants, females produce an ovum and males produce pollen. Plants produce gametes through mitosis in gametophytes.

As used herein, “gametocide” refers any substance that substantially eliminates the viability of a gamete of a plant. Such substances can include, but are not limited to, herbicides. Herbicides useful in the context of the claims include, but are not limited to, acetochlor, acifluorfen (and its sodium salt), aclonifen, acrolein (2-propenal), alachlor, alloxydim, ametryn, amicarbazone, amidosulfuron, aminopyralid, amitrole, ammonium sulfamate, anilofos, asulam, atrazine, azimsulfuron, beflubutamid, benazolin, benazolin-ethyl, bencarbazone, benfluralin, benfuresate, bensulfuron-methyl, bensulide, bentazone, benzobicyclon, benzofenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clethodim, clodinafop-propargyl, clomazone, clomeprop, clopyralid, clopyralid-olamine, cloransulam-methyl, CUH-35 (2-methoxyethyl 2-[[[4-chloro-2-fluoro-5-[(1-methyl-2-propynyl)oxy]phenyl](3-fluorobenzoyl)amino]carbonyl]-1-cyclohexene-1-carboxylate), cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate), halosulfuron-methyl, haloxyfop-etotyl, haloxyfop-methyl, hexazinone, HOK-201 (N-(2,4-difluorophenyl)-1,5-dihydro-N-(1-methylethyl)-5-oxo-1-[(tetrahydro-2H-pyran-2-yl)methyl]-4H-1,2,4-triazole-4-carboxamide), imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxyamid, phenmedipham, picloram, picloram-potassium, picolinafen, pinoxaden, piperofos, pretilachlor, primisulfuron-methyl, prodiamine, profoxydim, prometon, prometryn, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxsulam, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiocarbazil, topramezone, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate is disclosed.

Other suitable herbicides and agricultural chemicals are known in the art, such as, for example, those described in WO 2005/041654. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub. Combinations of various herbicides can result in a greater-than-additive (i.e., synergistic) effect on weeds and/or a less-than-additive effect (i.e. safening) on crops or other desirable plants. In certain instances, combinations of glyphosate with other herbicides having a similar spectrum of control but a different mode of action will be particularly advantageous for preventing the development of resistant weeds. Herbicidally effective amounts of any particular herbicide can be easily determined by one skilled in the art through simple experimentation.

Herbicides may be classified into groups and/or subgroups as described herein above with reference to their mode of action, or they may be classified into groups and/or subgroups in accordance with their chemical structure. Non-limiting examples of such groups include ALS Inhibitors, inhibitors of Acetyl CoA carboxylase (ACCase), inhibitors of Photosystem II, Photosystem-I-electron diversion compounds, inhibitors of PPO (protoporphyrinogen oxidase), bleaching compounds, inhibitors of EPSP Synthase, inhibitors of glutamine synthetase, inhibitors of DHP (dihydropteroate) synthase, microtubule assembly inhibitors, mitosis/microtubule organization inhibitors, cell division inhibitors, cell wall synthesis inhibitors, membrane disruptors, non-ACC inhibiting lipid synthesis inhibitors, synthetic auxins, auxin transport inhibitors, and others. Exemplary ALS inhibitors include Sulfonylureas (including, but not limited to, Azimsulfuron, Chlorimuron-ethyl, Metsulfuron-methyl, Nicosulfuron, Rimsulfuron, Sulfometuron-methyl, Thifensulfuron-methyl, Tribenuron-methyl, Amidosulfuron, Bensulfuron-methyl, Chlorsulfuron, Cinosulfuron, Cyclosulfamuron, Ethametsulfuron-methyl, Ethoxysulfuron, Flazasulfuron, Flupyrsulfuron-methyl, Foramsulfuron, Imazosulfuron, Iodosulfuron-methyl, Mesosulfuron-methyl, Oxasulfuron, Primisulfuron-methyl, Prosulfuron, Pyrazosulfuron-ethyl, Sulfosulfuron, Triasulfuron, Trifloxysulfuron, Triflusulfuron-methyl, Tritosulfuron, Halosulfuron-methyl, Flucetosulfuron), Sulfonylaminocarbonyltriazolinones (including, but not limited to, Flucarbazone and Procarbazone), Triazolopyrimidines (including, but not limited to, Cloransulam-methyl, Flumetsulam, Diclosulam, Florasulam, Metosulam, Penoxsulam, and Pyroxsulam), Pyrimidinyloxy(thio)benzoates (including, but not limited to, Bispyribac, Pyriftalid, Pyribenzoxim, Pyrithiobac, and Pyriminobac-methyl), Imidazolinones (including, but not limited to, Imazapyr, Imazethapyr, Imazaquin, Imazapic, Imazamethabenz-methyl, and Imazamox). Exemplary inhibitors of Acetyl CoA carboxylase (ACCase) include Aryloxyphenoxypropionates (“FOPs”) (including, but not limited to, Quizalofop-P-ethyl, Diclofop-methyl, Clodinafop-propargyl, Fenoxaprop-P-ethyl, Fluazifop-P-butyl, Propaquizafop, Haloxyfop-P-methyl, Cyhalofop-butyl, and Quizalofop-P-ethyl) and Cyclohexanediones (“DIMs”) (including, but not limited to, Alloxydim, Butroxydim, Clethodim, Cycloxydim, Sethoxydim, Tepraloxydim, and Tralkoxydim). Exemplary inhibitors of Photosystem II include Triazines (including, but not limited to, Ametryne, Atrazine, Cyanazine, Desmetryne, Dimethametryne, Prometon, Prometryne, Propazine, Simazine, Simetryne, Terbumeton, Terbuthylazine, Terbutryne, and Trietazine), Triazinones (including, but not limited to, Hexazinone, Metribuzin, and Metamitron), Triazolinones (including, but not limited to, Amicarbazone), Uracils (including, but not limited to, Bromacil, Lenacil, and Terbacil), Pyridazinones (including, but not limited to Pyrazon), Phenyl carbamates (including, but not limited to, Desmedipham and Phenmedipham), Ureas (including, but not limited to, Fluometuron, Linuron, Chlorobromuron, Chlorotoluron, Chloroxuron, Dimefuron, Diuron, Ethidimuron, Fenuron, Isoproturon, Isouron, Methabenzthiazuron, Metobromuron, Metoxuron, Monolinuron, Neburon, Siduron, and Tebuthiuron), Amides (including, but not limited to, Propanil and Pentanochlor), Nitriles (including, but not limited to, Bromofenoxim, Bromoxynil, and Ioxynil), Benzothiadiazinone (including, but not limited to, Bentazon), and Phenylpyridazines (including, but not limited to Pyridate and Pyridafol). Exemplary Photosystem-I-electron diversion compounds include Bipyridyliums (including, but not limited to, Diquat and Paraquat). Exemplary inhibitors of PPO (protoporphyrinogen oxidase) include Diphenylethers (including, but not limited to, Acifluorfen-Na, Bifenox, Chlomethoxyfen, Fluoroglycofen-ethyl, Fomesafen, Halosafen, Lactofen, and Oxyfluorfen), Phenylpyrazoles (including, but not limited to, Fluazolate and Pyraflufen-ethyl), N-phenylphthalimides (including, but not limited to, Cinidon-ethyl, Flumioxazin, and Flumiclorac-pentyl), Thiadiazoles (including, but not limited to, Fluthiacet-methyl and Thidiazimin), Oxadiazoles (including, but not limited to, Oxadiazon and Oxadiargyl), Triazolinones (including, but not limited to, Carfentrazone-ethyl and Sulfentrazone), Oxazolidinediones (including, but not limited to, Pentoxazone), Pyrimidindiones (including, but not limited to, Benzfendizone and Butafenicil), and others (including, but not limited to, Pyrazogyl and Profluazol). Exemplary bleaching compounds include Pyridazinones (including, but not limited to, Norflurazon), Pyridinecarboxamides (including, but not limited to, Diflufenican and Picolinafen), Triketones (including, but not limited to, Mesotrione and Sulcotrione), Isoxazoles (including, but not limited to, Isoxachlortole and Isoxaflutole), Pyrazoles (including, but not limited to, Benzofenap, Pyrazoxyfen, and Pyrazolynate), Triazoles (including, but not limited to, Amitrole), Isoxazolidinones (including, but not limited to, Clomazone), Ureas (including, but not limited to, Fluometuron), Diphenylethers (including, but not limited to, Aclonifen), and others (including, but not limited to, Beflubutamid, Fluridone, Flurochloridone, Flurtamone, and Benzobicyclon). Exemplary inhibitors of EPSP Synthase include Glycines (including, but not limited to, Glyphosate and Sulfosate). Exemplary inhibitors of glutamine synthetase include Phosphinic Acids (including, but not limited to, Glufosinate-ammonium and Bialaphos). Exemplary inhibitors of DHP (dihydropteroate) synthase include Carbamates (including, but not limited to, Asulam). Exemplary microtube assembly inhibitors include Dinitroanilines (including, but not limited to Benfluralin, Butralin, Dinitramine, Ethalfluralin, Oryzalin, Pendimethalin, and Trifluralin), Phosphoroamidates (including, but not limited to, Amiprophos-methyl and Butamiphos), Pyridines (including, but not limited to, Dithiopyr and Thiazopyr), Benzamides (including, but not limited to, Pronamide and Tebutam), and Benzenedicarboxylic acids (including, but not limited to, Chlorthal-dimethyl). Exemplary mitosis/microtubule organization inhibitors include Carbamates (including, but not limited to, Chlorpropham, Propham, and Carbetamide). Exemplary cell division inhibitors include Chloroacetamides (including, but not limited to, Acetochlor, Alachlor, Butachlor, Dimethachlor, Dimethanamid, Metazachlor, Metolachlor, Pethoxamid, Pretilachlor, Propachlor, Propisochlor, and Thenylchlor), Acetamides (including, but not limited to, Diphenamid, Napropamide, and Naproanilide), Oxyacetamides (including, but not limited to, Flufenacet and Mefenacet), Tetrazolinones (including, but not limited to, Fentrazamide), and others (including, but not limited to, Anilofos, Cafenstrole, Indanofan, and Piperophos). Exemplary cell wall synthesis inhibitors include Nitriles (including, but not limited to, Dichlobenil and Chlorthiamid), Benzamides (including, but not limited to, Isoxaben), and Triazolocarboxamides (including, but not limited to, Flupoxam). Exemplary membrane disruptors include Dinitrophenols (including, but not limited to, DNOC, Dinoseb, and Dinoterb). Exemplary non-ACC inhibiting lipid synthesis inhibitors include Thiocarbamates (including, but not limited to, Butylate, Cycloate, Dimepiperate, EPTC, Esprocarb, Molinate, Orbencarb, Pebulate, Prosulfocarb, Benthiocarb, Tiocarbazil, Triallate, and Vernolate), Phosphorodithioates (including, but not limited to, Bensulide), Benzofurans (including, but not limited to, Benfuresate and Ethofumesate) and Halogenated alkanoic acids (including, but not limited to, TCA, Dalapon, and Flupropanate). Exemplary synthetic auxins include Phenoxycarboxylic acids (including, but not limited to, Clomeprop, 2,4-D, and Mecoprop), Benzoic acids (including, but not limited to, Dicamba, Chloramben, and TBA), Pyridine carboxylic acids (including, but not limited to, Clopyralid, Fluroxypyr, Picloram, Tricyclopyr), Quinoline carboxylic acids (including, but not limited to, Quinclorac and Quinmerac), and others (including, but not limited to, Benazolin-ethyl). Exemplary auxin transport inhibitors include Phthalamates (including, but not limited to, Naptalam and Diflufenzopyr-Na). Examples of other herbicides include Arylaminopropionic acids (including, but not limited to, Flamprop-M-methyl/-isopropyl), Pyrazolium (including, but not limited to, Difenzoquat), Organoarsenicals (including, but not limited to, DSMA and MSMA), and others (including, but not limited to, Bromobutide, Cinmethylin, Cumyluron, Dazomet, Daimuron-methyl, Dimuron, Etobenzanid, Fosamine, Metam, Oxaziclomefone, Oleic acid, Pelargonic acid, Pyributicarb). Other herbicides may also be used in connection with the claimed method, whether currently known or as-yet undeveloped. In addition, combinations of herbicides may be used in the context of the claims. As used herein, a “herbicide tolerance gene” is a gene conferring partial or complete tolerance of a plant to a particular herbicide. Herbicide tolerance genes that may be used in the context of the claimed invention include, but are not limited to, a gene encoding glyphosate acetyltransferase as described more fully in U.S. Pat. Nos. 7,405,074 and 7,462,481 and U.S. App. Publications 2003/0083480, 2004/0082770, 2005/0246798 and 2007/0061917, each of which is herein incorporated by reference in their entirety; a gene that encodes a glyphosate oxido-reductase enzyme as described more fully in U.S. Pat. Nos. 5,776,760 and 5,463,175; polynucleotides that confer on the plant the capacity to produce a higher level of 5-enolpyruvylshikimate-3-phosphate synthase (EPSPS), for example, as more fully described in U.S. Pat. Nos. 6,248,876 B1; 5,627,061; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E; and 5,491,288; and international publications WO 97/04103; WO 00/66746; WO 01/66704; and WO 00/66747, further including gdc-1 (U.S. App. Publication 20040205847); EPSP synthases with class III domains (U.S. App. Publication 20060253921); gdc-1 (U.S. App. Publication 20060021093); gdc-2 (U.S. App. Publication 20060021094); gro-1 (U.S. App. Publication 20060150269); grg23 or grg 51 (U.S. App. Publication 20070136840); GRG32 (U.S. App. Publication 20070300325); GRG33, GRG35, GRG36, GRG37, GRG38, GRG39 and GRG50 (U.S. App. Publication 20070300326), U.S. App. Publication 20040177399; 20050204436; 20060150270; 20070004907; 20070044175; 2007010707; 20070169218; 20070289035; and, 20070295251, each of which is herein incorporated by reference in their entirety; genes conferring tolerance to sulfonylurea and/or imidazolinone, for example, as described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, each of which is herein incorporated by reference in their entirety; additional EPSPS sequences that are tolerant to glyphosate, such as those described in U.S. Pat. Nos. 6,248,876; 5,627,061; 5,804,425; 5,633,435; 5,145,783; 4,971,908; 5,312,910; 5,188,642; 4,940,835; 5,866,775; 6,225,114; 6,130,366; 5,310,667; 4,535,060; 4,769,061; 5,633,448; 5,510,471; Re. 36,449; RE 37,287 E; and 5,491,288; and international publications WO 97/04103; WO 00/66746; WO 01/66704; and WO 00/66747; U.S. Pat. Nos. 6,040,497; 5,094,945; 5,554,798; 6,040,497; Zhou et al. (1995) Plant Cell Rep. :159-163; WO 0234946; WO 9204449; U.S. Pat. Nos. 6,225,112; 4,535,060, and 6,040,497, which are incorporated herein by reference in their entireties for all purposes; genes conferring tolerance to 2,4-D and other phenoxy auxin herbicides, aryloxyphenoxypropionate herbicides, pyridyloxyacetate herbicides and aryloxyphenoxyalkanecarboxylic acid herbicides as described more fully in U.S. Pat. Nos. 6,414,222, 5,808,174, 5,623,782, U.S. App. Publication 2009/0093366 and 2003/0154507 and international publications WO 2007/053482, WO 2005/107437, and WO 2008/141154 each of which is herein incorporated by reference in their entirety; genes conferring tolerance to auxin-like herbicides and dicamba as described more fully in U.S. Pat. Nos. 7,105,724, 7,022,896, 6,153,401, 6,100,446 and U.S. App. Publications 2009/0081760, 2008/0120739, 2008/0119361, 2006/0168700, US 2003/0135879, 2003/0115626, genes conferring tolerance to the herbicides glufosinate and bialaphos as described more fully in U.S. Pat. Nos. 5,273,894, 5,276,268, 5,637,489 and 7,112,665 which are incorporated herein by reference in their entireties for all purposes, genes conferring tolerance to hydroxy phenyl pyruvate dioxygenase (HPPD) inhibitors, such as the herbicides of the family of isoxazoles or of that of the triketones or the pyrazolinates as described more fully in U.S. Pat. Nos. 7,304,209, 7,250,561, 6,768,044, 6,268,549, 6,245,968, 6,087,563, 7,297,541, 7,312,379, and U.S. App. Publication 2008/0127371 which are incorporated herein by reference in their entireties for all purposes, genes conferring tolerance to PPO inhibitory herbicides as described more fully in U.S. Pat. Nos. 5,767,373, 5,939,602, 6,023,012, 6,084,155 and U.S. App. Publication 2005/0081259 which are incorporated herein by reference in their entireties for all purposes. Other EPSPS events of interest include MON-89788-1 (MON89788). Other herbicide tolerance events and/or genes may also be used, whether currently-known or yet to be discovered. Further, combinations of herbicide tolerance events and/or genes may also be used in the context of the claims.

As used herein, “genotype” refers to the entire genetic constitution of a cell or organism. The entirety of all alleles at all loci constitutes a genotype.

As used herein, “germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

As used herein, “heterogeneity” is used to indicate that individuals within the group differ in genotype at one or more specific loci. In contrast, as used herein, the term “homogeneity” indicates that members of a group have the same genotype at one or more specific loci.

As used herein, “heterozygous” refers to an individual with more than one allele type present at a given locus (e.g., a diploid individual with one copy each of two different alleles).

As used herein, “homozygous” refers to an individual with only one type of allele at a given locus (e.g., a diploid individual has a copy of the same allele at a locus for each of two homologous chromosomes). The term is used when alleles in homologous chromosomes are identical at a particular locus.

As used herein, “inbred” refers to a line developed through inbreeding or doubled haploidy that preferably comprises homozygous alleles at about 95% or more of its loci.

As used herein, the term “plant” includes reference to an immature or mature whole plant, including a plant that has been detasseled or from which seed or grain has been removed. Seed or embryo that will produce the plant is also considered to be the plant.

As used herein, the term “plant parts” includes leaves, stems, roots, seed, grain, embryo, pollen, ovules, flowers, ears, cobs, husks, stalks, root tips, anthers, pericarp, silk, tissue, cells and the like.

As used herein, the term “transgenic” includes any cell, cell line, callus, tissue, plant part, or plant, the genotype of which has been altered by the presence of a heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

As used herein, the term “transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette.

The methods of the present invention may be applied in several ways in order to integrate a transgene of interest into highly yielding inbreds and hybrids with acceptable agronomics and other traits desired by crop growers. The methods of the present invention result in inbred lines with improved trait purity. These methods may be used for maize inbreds, for example, which must be: homogenous such that virtually all inbreds have the same genotype; essentially homozygous having from about 95% to 100% of its genetic loci with the presence of one form of allele; and essentially true breeding. This is distinguishable from a hybrid with is significantly heterozygous.

In one embodiment of the present invention, a herbicide is used as the gametocide to select cells that lack the particular herbicide resistance in order to produce herbicide resistant seed and crops. This results in a decrease of herbicide susceptible seed and crops. The methods include applying a herbicide, such as glyphosate, late to hemizygous transgenic plants during germ cell development. This involves contacting an inbred line that is hemizygous for a transgene encoding for herbicide resistance (or any other identified gametocide resistance) with the herbicide (or other gametocide). Such hemizygous plants are 1N for the transgene. After meiosis, one-half of the germ cells have the transgene and one-half lack the transgene. All other cells in the plant contain the transgene.

Application of herbicide after meiosis kills the germ cells lacking the transgene. In one embodiment, the herbicide is applied to both inbred lines planted in a production field after pollen development. The timing of the herbicide application results in the death of the herbicide-susceptible inbred line. The pollen from the herbicide-susceptible inbreds is still available to pollinate the female gametes of the herbicide-resistant inbred line. Therefore, only those plants with cells containing the transgene of interest survive the herbicide application.

Similarly, if multiple transgenes are being introduced to the inbred line, one or more gametocides may be used to select for only the gametes that contain the multiple transgenes. By way of non-limiting example, to the extent an inbred line is hemizygous for each of two transgenes, one encoding for tolerance to a first gametocide and the other for a second gametocide, application of the two gametocides to the gametes will select for those gametes that contain both transgenes. Alternatively, if two transgenes are required to confer tolerance to the gametocide, a single gametocide may be used to select gametes having both transgenes. More than two transgenes and more than two gametocides may also be used.

These methods result in a breeding program with selfed plants after treatment being homozygous (2N) for the transgene (or transgenes) in only one generation. If the transgene is part of a construct containing other desirable transgenes, the linkage will also allow a breeder to transition from hemizygous to homozygous lines in one generation.

This invention is also of value in seed production. Parent seed must increase parent lines that are pure lines, both in the traditional sense and for the transgenes they contain. Inbred corn lines need to be nearly 100% homozygous for transgenes to produce hybrids that express the transgenic traits at sufficiently high levels to successfully market the seed. Application of the proper chemical to select against the germ cells that lack the transgene will enable all seed that self pollinates in that increase field to be homozygous for the transgenic trait. Again, if the transgene is linked to other transgenic traits, after treatment, the inbred line will be homozygous for the entire construct.

These methods are similar to laboratory single cell selection in Petri dishes using antibiotics or other selective lethal agents. Such methods allow the selection of identifiable cells, or in the case of the methods of the present invention, germ cells lacking a transgene of interest are selected. Accordingly, one of the applications of this novel approach is to ensure transgenetic purity in inbred line development.

The specific selection of female gametes is dependent upon the application timing of the particular gametocide. In one embodiment of the invention, wherein glyphosate was applied to maize plants, the chemical selection of female gametes not carrying the transgene conferring glyphosate tolerance is achieved due to the later application of the glyphosate. Progeny from applications at the V10 to V14 growth stages and more preferably at the V15 to V19 growth stages were significantly more resistant to glyphosate than under previous attempts (see for example, Kaster et al. 2004; Thomas et al. 2004). Wild type ovules can be rouged with, for example, glyphosate provided the rates are high enough and application timings follow the methods of the present invention, resulting in improved seed purity and improved processes of trait integration.

Additionally, fewer wild type kernels and ears during backcrossing reduce the number of wild type rows in the next generation of selfing. For parent seed increases, spray applications during the V10 to V14 or the V15 to V19 stages ensure that unwanted wild type gametes are not passed into planting seed.

All publications and patent applications in this specification are indicative of the level of ordinary skill in the art to which this invention pertains. All publications and patent applications are herein incorporated by reference to the same extent as if each individual publication or patent application was specifically and individually indicated by reference. The disclosure of each reference set forth herein is incorporated herein by reference in its entirety.

### EXAMPLES

This invention can be better understood by reference to the following examples. The foregoing and following description of the present invention and the various embodiments are not intended to limit the invention, but rather are illustrative thereof. Therefore, it will be understood that the invention is not limited to the specific details of these examples. It will be appreciated by those skilled in the art that other embodiments of the invention may be practiced without departing from the spirit and the scope of the invention, the scope of which is defined by the appended claims.

### Example 1

The elimination of non-transgenic gametes was also tested at the V12-V19 stages. Meiosis sorted the transgene of a diploid hemizygous microsporophyte or macrosporophyte into one-half of the gametes. The resulting transgenic construct contained a gene conferring plant resistance or tolerance to a specific herbicide, namely glyphosate. Application of the herbicide treatments at various growth phases eliminated gametes lacking the transgenic construct.

Glyphosate treatments included: V12, V17, V19, V15+V17, and V17+V19. Each application, included splits 3 quarts/A. Only resistant plants were sprayed. Glufosinate treatments included: V12, V17, V18, V19, V15+V17, and V17+V19. Each application, included splits 1.5 quarts/A. Only resistant plants were sprayed.

Pollination of the sprayed resistant plants included: 4 selfed, 4 pollinations from unsprayed resistant, 4 pollinations from susceptible. Pollination of the unsprayed resistant plants included: 4 selfed, 4 pollinations from sprayed resistant, 4 pollinations from susceptible. Further, pollination of the susceptible plants included: 4 selfed, 4 pollinations from sprayed resistant, 4 pollinations from unsprayed resistant. There were two replications. The seed was harvested at maturity and germinated in growth chambers. Seedlings were treated with 2% v/v glyphosate or 3% glufosinate.

Survivors were counted and chi square analysis performed to determine whether segregation ratios deviated from the null hypothesis (e.g., 75:25 resistant phenotype for selfs or crosses within the resistant hemizygous hybrids, 50:50 resistant phenotype for crosses between resistant and susceptible hybrids, or 100% mortality for susceptible selfed seed).

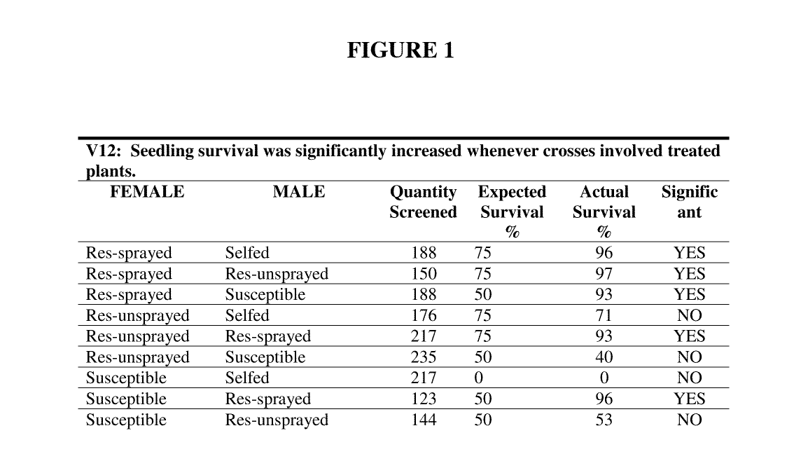

Application of glyphosate to glyphosate resistant plants at V12 eliminated most non-transgenic female and male gametes (FIG. 1).

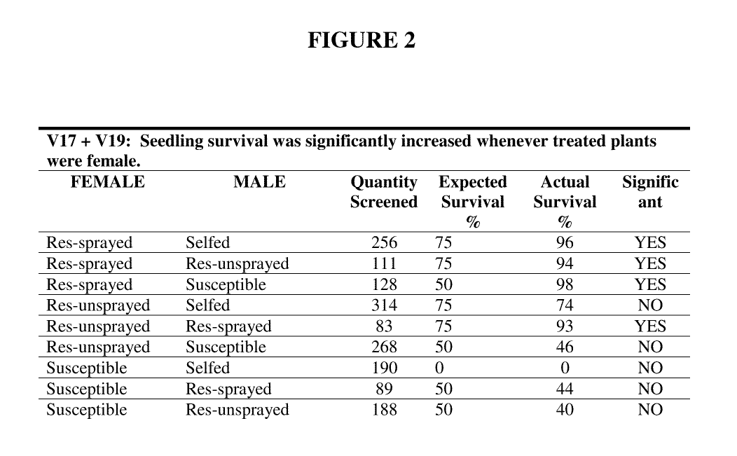

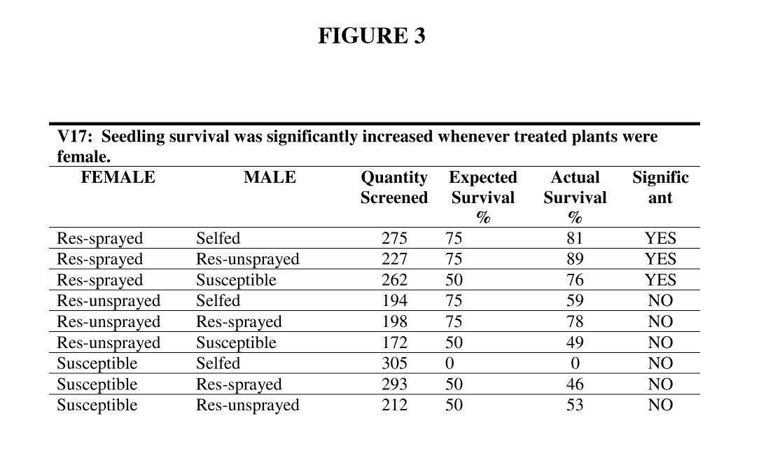

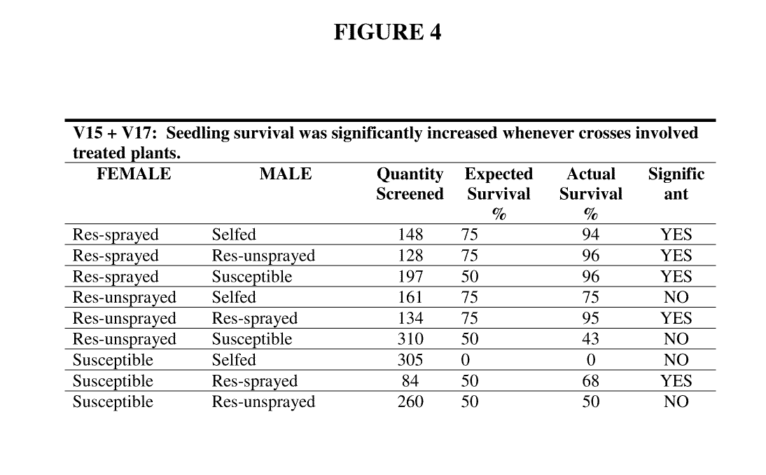

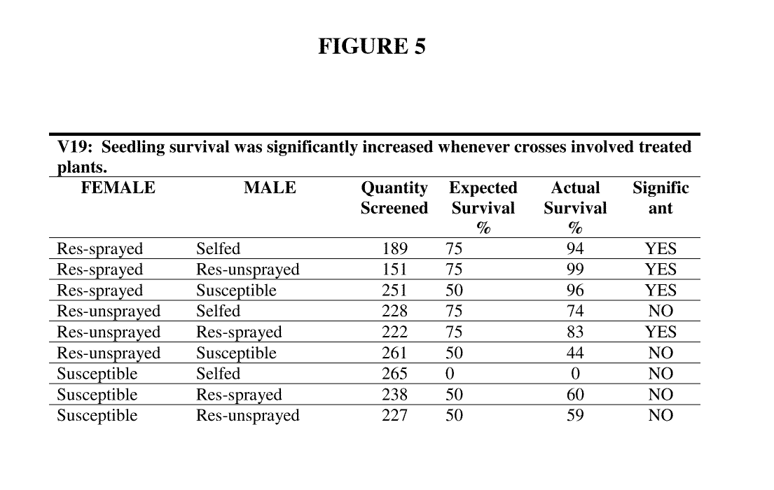

Application of glyphosate to glyphosate resistant plants at V15 or later primarily eliminated non-transgenic female gametes (FIGS. 2-5), illustrating the ability to eliminate non-transgenic gametes to enhance product quality by assuring all seed of a parent inbred is homozygous for a particular trait. Hybrids produced with such parent uniformly express all traits linked to or included in the genetic construct.

### Example 2

Four row plots of a heterozygous inbred for glyphosate resistance were established. At the V10, V12, V14 and V10+V14 stages of plant development, glyphosate was broadcast to the center two rows. The WeatherMax glyphosate formulation was applied at 0, 1.6 and 3.2 l/Ha, such rates corresponding to 0, 1 and 2 times the normal commercial rates in corn production. To assure there was no glyphosate drift on the non-treated controls, non-treated plants were separated by 16 border rows.

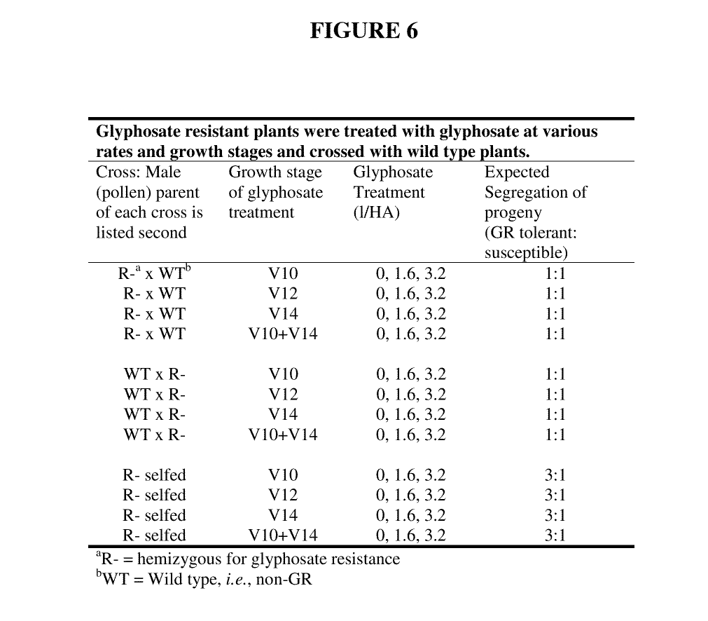

A series of crosses were made from the treated plants to identify the gamete rouging effects of glyphosate on both pollen and ovules. To characterize the effects on pollen, heterozygous plants treated with glyphosate were crossed onto a wild type inbred. To characterize the effect of glyphosate on ovules, non-treated wild type pollen was crossed to heterozygous plants treated with glyphosate. The various crosses and the expected outcomes are listed in FIG. 6. Finally, the treated heterozygous plants were selfed and tested for zygosity via quantitative PCR.

### Example 3

The progeny of the various crosses from Example 2 were evaluated by two methods. In the first method, 25 seeds per replication (4 replications in total) were planted into rag dolls and grown at 25° C. in darkness four days. After this time, the rag dolls were transferred to a greenhouse to allow the shoots to turn green. After four days in the greenhouse, seedlings for each rag doll were dipped in a 2% v/v solution of RoundUp Ultra (concentration corresponding to common concentrations in spray applications at the field level). After one week, individual plants were rated as normal, dead or abnormal. Normal plants were assumed to carry the gene for tolerance to glyphosate, dead plants were assumed to be wild type.

In the second method of analysis, the glyphosate resistant selfed plants were grown in the field and leaf punched at the V8 stage of development. Leaf tissues were then frozen and scored for their level of zygosity according to standard PCR protocols for glyphosate resistance. The data from the progeny tests was arc sine transformed and submitted to a common analysis of variance. Since the F-test for overall treatments was highly significant, tests of significance between the controls and the various treatments were also conducted.

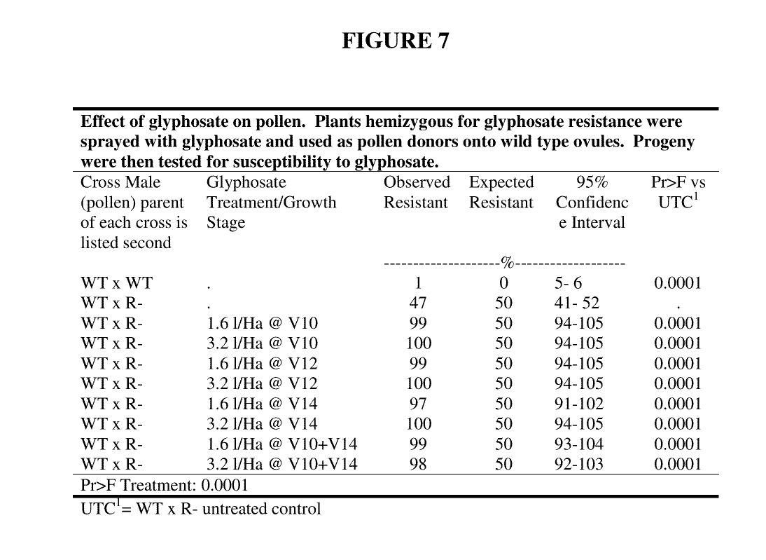

The crosses separated the effects of glyphosate on pollen and ovule gametes. To separate effects on pollen, heterozygous plants were sprayed with glyphosate and pollen collected from treated plants was crossed onto homozygous wild type plants. If glyphosate had no affect on wild type pollen the segregation ratios of the progeny from this cross would be 50% resistant to glyphosate and 50% susceptible to glyphosate. The leaf dip test indicates that glyphosate was highly effective in rouging wild type pollen, as normal expected segregation ratios were disturbed (as expressed in FIG. 7).

For instance, progeny from non-treated plants crossed onto wild type ovules segregated at 47%, statistically equal with expected segregation rate of 50%. Progeny of plants treated with glyphosate and crossed to wild type ovules were all nearly 100% resistant to glyphosate. The effect of glyphosate on wild type pollen was not affected by rate or time of application. Applications at V10+V14 (providing two-pass control) were no more effective than a single application.

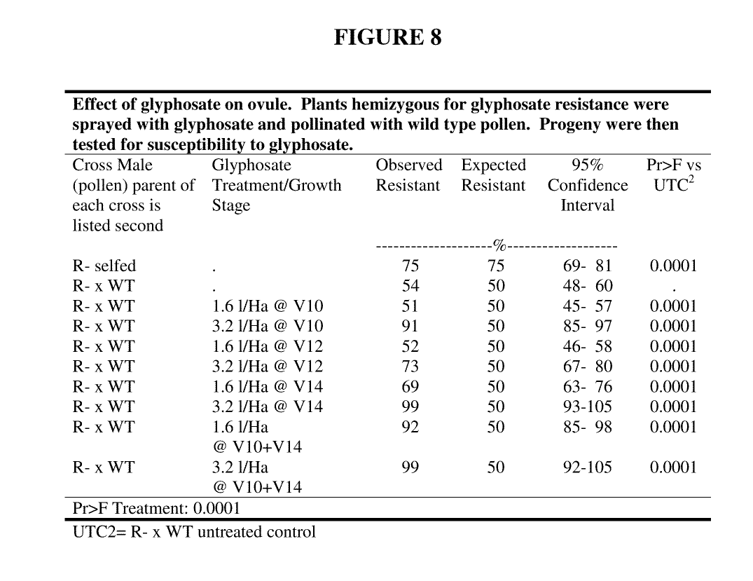

The effects of glyphosate on wild type ovules were less pronounced than for pollen gametes (as expressed in FIG. 8). In the control treatment, untreated heterozygous plants were pollinated with homozygous wild type pollen. Expected segregation ratios for the progeny of this cross would be 50% resistant and 50% susceptible. Here, the control progeny segregated at 54% resistant, statistically equal to the expected ratio of 50%. Progeny of plants treated at V10 and V12 at the lowest rate of glyphosate segregated as expected if glyphosate had no affect on wild type ovules. At the highest rate of glyphosate, the segregation of progeny was clearly disturbed. High rates applied at the V14 stage produced progeny nearly 100% resistant, while applications made before V14 were intermediate. The two pass program produced progeny nearly 100% resistant, but this program was not any more effective than a single spray application at V14.

### Example 4

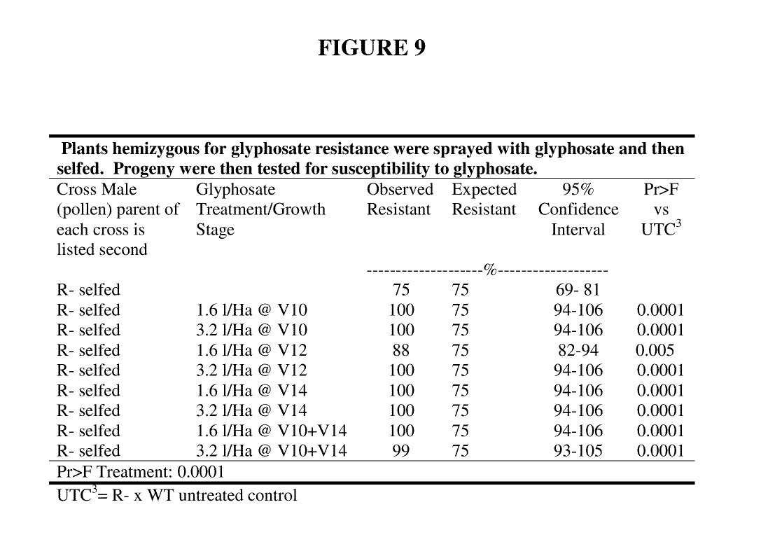

Heterozygous plants were also selfed. The progeny of non-treated selfed plants segregated as expected at 75% resistant and 25% susceptible (as expressed in FIG. 9). Except for plants treated at V12 with the lowest rate of glyphosate, all the other treatments produced progeny nearly 100% resistant to glyphosate, indicating significant rouging of wild type gametes.

### Example 5

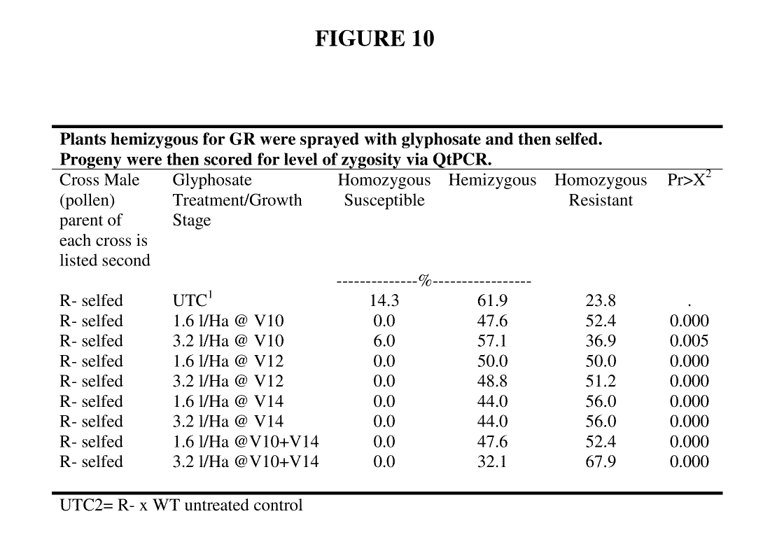

Plants hemizygous for glyphosate resistance were sprayed with glyphosate and then selfed. The progeny were then scored for level of zygosity via QtPCR. While the results from PCR corroborate data from the leaf dip assays, the magnitude of the response is less (as expressed in FIG. 10). Applications of glyphosate significantly reduced the ratio of homozygous wild type plants compared to plants which were selfed and treated with glyphosate. Two pass applications at the highest rates of glyphosate produced the greatest percentage of homozygous glyphosate resistant plants.

What is claimed is:

1. A method of producing maize lines with high trait purity comprising:
applying a gametocide to a population of plants, wherein some of said plants are hemizygous for resistance to said gametocide, wherein said gametocide effectively eliminates the viability of substantially all female gametes not carrying the gene conferring gametocide resistance; and
producing seed or progeny from said seed, wherein said seed or progeny are substantially homozygous for resistance to said gametocide.

2. The method of claim 1 wherein the seed or progeny are hybrids.

3. The method of claim 1 wherein said herbicide is glyphosate.

4. The method of claim 1 wherein said gametocide is applied more than once during the life of the plant.

5. The method of claim 1 wherein said gametocide is applied after meiosis.

6. The method of claim 1 wherein said gametocide is applied after pollen development.

7. The method of claim 1 wherein said gametocide is applied during one or more of growth stages V10 to V14.

8. The method of claim 1 wherein said gametocide is applied during one or more of growth stages V15 to V19.

9. The method of claim 1 wherein wherein said seed or progeny substantially homozygous for resistance to said gametocide is produced on a commercial scale.

10. The method of claim 1 wherein said seed or progeny substantially homozygous for resistance to said gametocide are produced in a single generation.

11. A method for increasing the efficiency of a breeding program comprising:
applying a herbicide to a population of maize plants, wherein some of said plants are hemizygous for resistance to said herbicide, wherein said herbicide effectively eliminates the viability of substantially all female gametes not carrying the gene conferring resistance to said herbicide; and
producing seed or progeny from said seed, wherein said seed or progeny are substantially homozygous for resistance to said herbicide.

12. The method of claim 11 wherein the seed or progeny are hybrids.

13. The method of claim 11 wherein said gametocide is applied after meiosis.

14. The method of claim 11 wherein said gametocide is applied after pollen development.

15. The method of claim 11 wherein said herbicide is glyphosate.

16. The method of claim 11 wherein said herbicide is applied more than once during the life of said plant.

17. The method of claim 11 wherein said herbicide is applied during one or more of growth stages V10 to V14.

18. The method of claim 11 wherein said herbicide is applied during one or more of growth stages V15 to V19.

19. The method of claim 11 wherein said seed or progeny substantially homozygous for resistance to said herbicide are produced on a commercial scale.

20. The method of claim 11 wherein said seed or progeny substantially homozygous for resistance to said herbicide are produced in a single generation.
